(19) United States
(12) Patent Application Publication
(10) Pub. No.: US 2010/0063017 A1
(43) Pub. Date: Mar. 11, 2010

(54) Ophthalmic Suspension for Ocular Use

(76) Inventor: Kothanda Raman T. Rajan, Bangalore (IN)

Correspondence Address: ALLERGAN, INC., 2525 DUPONT DRIVE, T2-7H, IRVINE, CA, 92612-1599, US

(21) Appl. No.: 12/554,514

(22) Filed: Sep. 4, 2009

Related U.S. Application Data
(60) Provisional application No. 61/095,452, filed on Sep. 9, 2008.

(51) Int. Cl. A61K 31/573; A61P 27/02

(52) U.S. Cl. 514/171

(57) ABSTRACT

A gatifloxacin and prednisolone topical ophthalmic pharmaceutical compositions for prevention and treatment of ophthalmic bacterial infections and inflammatory conditions associated with pre-surgical and/or post surgical ocular surgeries.

### RELATED APPLICATIONS

This application claims priority to U.S. Provisional Patent Application No. 61/095,452, filed on Sep. 9, 2008, the entire disclosure of which is incorporated herein by this reference.

### FIELD OF THE INVENTION

The present invention is directed to a gatifloxacin and prednisolone topical antibiotic pharmaceutical compositions for prevention and treatment of ophthalmic bacterial infections, particularly bacterial infections in conjunction with inflammatory conditions associated with pre-surgical and/or post surgical ocular surgeries.

### BACKGROUND OF THE INVENTION

Quinolone antibiotics are frequently used to prevent and treat ophthalmic infections and in general represents the current state of the art in the field of ophthalmic pharmaceutical compositions and methods of treatment. Some quinolone antibiotic compositions are effective in treating ophthalmic infections and have distinct advantages over prior ophthalmic antibiotic compositions, particularly those having relatively limited spectrums of antimicrobial activity. For example, neomycin, polymyxin B, gentamicin and tobramycin are primarily useful only against gram negative bacteria while bacitracin, gramicidin, and erythromycin are primarily useful only against gram positive bacteria. Thus quinoline antibiotics have advantages over many other classes of antibiotics in that they are broad spectrum. However, despite the general efficacy of available ophthalmic antibacterial compositions, there exists a strong need for improved quinoline ophthalmic antibacterial composition and methods of treatments of ophalmic infections, particularly those associated with inflammatory conditions, which are more effective than existing ophthalmic antibacterial compostions in treating key ophthalmic bacteria and which are less prone to the development of resistance by those bacteria. There is also a need for an ophthalmic composition which is useful in treating or preventing bacterial infection and inflammation both before and after eye surgery.

Thus it is an object of the invention to provide a topical ophthalmic preparation consisting of a fourth generation quinolone as anti-infective agent and an anti-inflammatory agent;

It is a further object of the invention to provide an ophthalmic composition comprising an anti-infective agent and an anti-inflammatory agent in pharmaceutically acceptable excipients and vehicle with an acceptable pH and viscocity;

It is a further object of the invention to provide a clear, stable aqueous suspension composition of an anti-infective and a steroidal anti-inflammatory which when administered topically to the eye, does not cause any irritation/discomfort to the eye; and,

It is a further object to provide aforesaid ophthalmic composition having more patient compliance and acceptability.

### SUMMARY OF INVENTION

The present invention is directed to a unique and efficacious combination of gatifloxacin and prednisolone for the treatment of ophthalmic bacterial infections and for the prevention of inflammation. The gatifloxacin and prednisolone compositions of the present invention are useful in both the treatment and prophylaxis of patients undergoing ophthalmic surgical procedures and may be used pre- or post-operatively to prevent or treat both inflammation and bacterial infection.

Ophthalmic infections are frequently accompanied by inflammation of the infected ophthalmic tissues and sometimes the surrounding tissues. Similarly, ophthalmic surgical procedures that pose a risk of microbial infections may also cause inflammation of the affected tissues. Thus, there is a need for ophthalmic pharmaceutical compositions which combine the anti-infective activity of one or more broad spectrum antibiotics with the anti-inflammatory properties of one or more steroidal or non-steroidal agents in a single composition.

Inflammatory disease is the third most frequent eye perturbation (after refraction error and dry eye syndrome) and topical corticosteroids for treatment of inflammatory conditions are well accepted among practitioners. Inflammatory conditions caused by invasive surgical procedures to the eye must also be considered, such as those caused by ophthalmic surgeries, in which the presence of infection in the post-op period is a constant concern by ophthalmologists. There is also the concern of inflammatory conditions which may exist pre-operatively which may be associated with a bacterial infection which will worsen post-operatively. When these infections are caused by virulent bacteria, it is possible that these bacterial infections can spread to other eye tissues/structures leading to sight threatening conditions.

Most cases of bacterial conjunctivitis are caused by Streptococcus pneumoniae, Haemophilus influenzae and Streptococcus sp. The prophylactic use of antibiotics both before and after invasive surgical procedures of the eye is an accepted therapeutic treatment and widely used in ophthalmology, as is documented in the literature. The constant search for new and more powerful broad spectrum antibiotic treatments has been encouraged in large part to the capacity of these microorganisms to develop resistance to antibiotics, and gatifloxacin is ideal for this purpose. The presence of prednisolone in the product's formulation complements the anti-bacterial action of gatifloxacin due to its anti-inflammatory effects, and contributes to the efficacy of gatifloxacin in the treatment and/or prevention of eye infections with a critical inflammatory component.

The present invention is directed to a gatifloxacin and prednisolone composition in the form of a sterile solution for ophthalmic topical use. The use of a combination drug with an anti-infective component is indicated where the risk of infection is high or where there is an expectation that potentially dangerous numbers of bacteria will be present in the eye. The anti-inflammatory component of the composition is useful in treating inflammation associated with physical trauma to ophthalmic tissues, inflammation associated with bacterial infections and inflammation resulting from surgical procedures. The combination of gatifloxacin and prednisolone is also useful in post-operative inflammation where there is an increased chance of bacterial infection. The compositions of the invention may also be used prophylactically in connection with various ophthalmic surgical procedures that create a risk of bacterial infection. Other examples of ophthalmic conditions which may be treated with the compositions of the present invention include infective conditions with associated inflammation and where the use of steroid is acceptable; such conditions may include, but not limited to conjunctivitis, keratitis, blepharitis, dacyrocystitis, hordeolum and corneal ulcers.

The composition of the present invention contains gatifloxacin in ranges of 0.01% w/v-1.5% w/v, 0.05% w/v-1.0% w/v; 0.1% w/v-1.0% w/v. 0.1% w/v-0.5% w/v but most preferable concentration of gatiflaxocin is 0.3% w/v. The composition of the present invention also contains prednisolone acetate in ranges between 1-20 mg/ml including 0.01% w/v-2.0% w/v, 0.05% w/v-1.5% w/v, 0.1% w/v-1.5% w/v but most preferably USP 10 mg (1.0% w/v). In combination, the most preferable concentration of gatifloxacin and prednisolone is 0.3% w/v of gatifloxacin to 1.0% w/v of prednisolone.

The gatifloxacin/prednislone composition of the present invention is specially formulated for topical application to ophthalmic tissues. The composition is sterile and has physical properties which are ideally suited for application to ophthalmic tissues, including tissues that have been compromised as the result of preexisting disease, trauma, surgery or other physical conditions.

The antibiotic concentration of gatifloxacin of 0.01% w/v-1.5% w/v and in particular 0.3% w/v in the present composition contains an ideal amount of gatifloxacin sufficient to provide a concentration in the aqueous humor and lacrimal fluid of the eye equal to or greater than the MIC90 (minimum inhibitory concentration levels to inhibit 90% growth) relative to gram-negative and gram-positive organisms commonly associated with ophthalmic infections. This amount is referred to as “an antimicrobial effective concentraion”. Gatifloxacin works by inhibiting DNA gyrase and topoisomerase IV both of which are necessary for the replication of bacteria.

TABLE I
Gatifloxacin Microbiology:

| | |
|---|---|
| Gatifloxacin has been | Aerobic Gram-Positive Bacteria |
| shown to be active against | Corynebacterium propinquum, Staphylococcus aureus, |
| most strains of the | Staphylococcus epidermidis, Streptococcus mitis, |
| following organisms both | Streptococcus pneumoniae |
| invitro and clinically, in | Aerobic Gram-Negative Bacteria |
| conjunctival infections | Haemophilus influenzae |
| Gatifloxacin exhibits in | Aerobes, Gram-Positive |
| vitro minimal inhibitory | Listeria monocytogenes, Staphylococcus saprophyticus, |
| concentrations (MICs) of | Streptococcus agalactiae, Streptococcus pyogenes, |
| 2 μg/mL or less (systemic | Streptococcus viridans Group, Streptococcus Groups C, |
| susceptible breakpoint | F, G |
| against most >90%) strains | Aerobes, Gram-Negative |
| of the following ocular | Acinetobacter lwoffii, Enterobacter aerogenes, |
| pathogens | Enterobacter cloacae, Escherichia coli, Citrobacter |
| | freundii Citrobacter koseri, Haemophilus parainfluenzae, |
| | Klebsiella oxytoca, Klebsiella pneumoniae, Moraxella |
| | catarrhalis, Morganella morganii, Neisseria gonorrhoeae, |
| | Neisseria meningitides, Proteus mirabilis, Proteus |
| | vulgaris, Serratia marcescens, Vibrio cholerae, Yersinia |
| | enterocolitica |
| | Other Microorganisms |
| | Chlamydia pneumoniae, Legionella pneumophila, |
| | Mycobacterium marinum, Mycobacterium fortuitum |
| | Mycoplasma pneumoniae |
| | Anaerobic Microorganisms |
| | Bacteroides fragilis, Clostridium perfringens |

The composition of the present invention also contains prednisolone acetate in ranges between 1-20 mg/ml but most preferably USP 10 mg (1.0% w/v) as an anti-inflammatory agent. The anti-inflammatory agent utilized in the present invention is broadly classified as steroidal. Prednisolone acetate is a glucocorticoid and has three to five times the anti-inflammatory potency of hydrocortisone. Glucocorticoides inhibit edema and fibrin disposition, phagocytic migration, capillary proliferation and deposition of collagen and scar tissue. The concentration of the anti-inflammatory agents contained in the composition of the present invention is based on the type of inflammation being treated. The concentration is sufficient to reduce inflammation in the targeted ophthalmic tissues following topical application of the compositions to those tissues. Such an amount is referred to as “an anti-inflammatory effective amount”.

One preferred embodiment of the present invention showing the concentrations of active ingredients is the following:

| | |
|---|---|
| Gatifloxacin sesquihydrate | 3 mg/ml |
| Prednisolone acetate USP | 10 mg/ml |
| Benzalkonium chloride | 0.05 mg/ml |
| Purified water | q.s. |

Another preferred embodiment of the invention if the following:

| | |
|---|---|
| Gatifloxacin | 3.0 mg/ml |
| Prednisolone acetate + | 10.000 mg/ml |
| 5% overage | |
| Methylcellulose 4MHPMC | 1.117 mg/ml |
| Sodium phosphate dibasic dihydrate | 8.720 mg/ml |
| Sodium phosphate monobasic | 5.250 mg/ml |
| Edetate disodium | 0.100 mg/ml |
| Hydrochloric acid 1 N | 0.020 mg/ml |
| Benzalkonium chloride (Solution 10%) | 0.050 mg/ml |
| Purified water | q.s. |

The composition of the current invention is administered to the affected ophthalmic tissues by topically applying one to four drops of the sterile suspension one to four times per day/eye or more as indicated.

Benzalkonium chloride is used as the preferred preservative in the present invention. However, it is possible that other preservatives commonly used in ophthalmic solutions be utilized such as Purite® (chlorine dioxide), polixetonium, polyhexamethylene biquanide, polyquad and sodium perborate.

Some embodiments of the present invention include:
1) A topical ophthalmic composition for treating or preventing ophthalmic bacterial infections in a human patient wherein the ophthalmic composition contains gatifloxacin and prednisolone.
2) The composition of paragraph 1 wherein the composition is also useful in preventing inflammation and bacterial infection in the eye following ophthalmic surgical procedures.
3) The composition of paragraphs 1 and 2 wherein the concentration of gatifloxacin is 0.01% to 1.5% w/v.
4) The composition of paragraphs 1-3 wherein the concentration of prednisolone is 0.01% w/v-2.0% w/v.
5) The composition of paragraph 3 wherein the concentration of gatifloxacin is 0.1% w/v-0.5% w/v.
6) The composition of paragraph 4 wherein the concentration of prednisolone is 0.1% w/v-1.5% w/v.
7) The composition of paragraph 5 wherein the concentration of gatifloxacin is 0.3% w/v.
8) The composition of paragraph 5 wherein the concentration of prednisolone is 1.0% w/v.
9) The composition of paragraphs 1-8 wherein the concentration of gatifloxacin is 0.1% w/v-0.5% w/v and the concentration of prednisolone is 0.1%-1.5% w/v.
10) The composition of paragraph 9 wherein the concentration of gatifloxacin is 0.3% w/v and the concentration of prednisolone is 1.0% w/v.
11) The composition of paragraph 10 further comprising benzalkonium chloride.
12) The composition of paragraph 11 wherein the benzalkonium chloride is present in the amount of 0.005% w/v.
13) The composition of paragraphs 10-12 wherein the pH is in the range of 6.5 to 7.4.
14) The composition of paragraph 1 and 10-13 wherein the composition has an osmotic value from 250 to 350 milliosmoles per kilogram of water.
15) The composition of paragraphs 1 and 10-14 wherein the composition is provided as a kit containing a 1-10 ml plastic dropper designed for topical administration of the composition.
16) The composition of paragraphs and 10-14 wherein the composition is provided as a kit containing a 5 ml plastic dropper designed for topical administration of the composition.
17) The composition of paragraphs 1 and 10-14 wherein the composition may be used for treatment of bacterial conjunctivitis by applying 1-4 drops of the composition to each eye per day.
18) The composition of paragraphs 1 and 10-14 wherein the composition may be used for prevention and treatment of inflammatory conditions following eye surgery by applying 1-4 drops of the composition to each eye per day.
19) The composition of paragraphs 1 and 10-14 wherein the composition may be used for prevention and treatment of inflammatory conditions before or following eye surgery by applying 1-4 drops of the composition to each eye per day.
20) The composition of paragraphs 1 and 10-14 wherein the composition may be used for prevention of bacterial conjunctivitis following eye surgery by applying 1-4 drops of the composition to each eye per day.

### BRIEF DESCRIPTION OF THE DRAWINGS

FIGS. 1A-1B show a basic manufacturing process for the gatifloxacin/prednislone composition of the present invention.

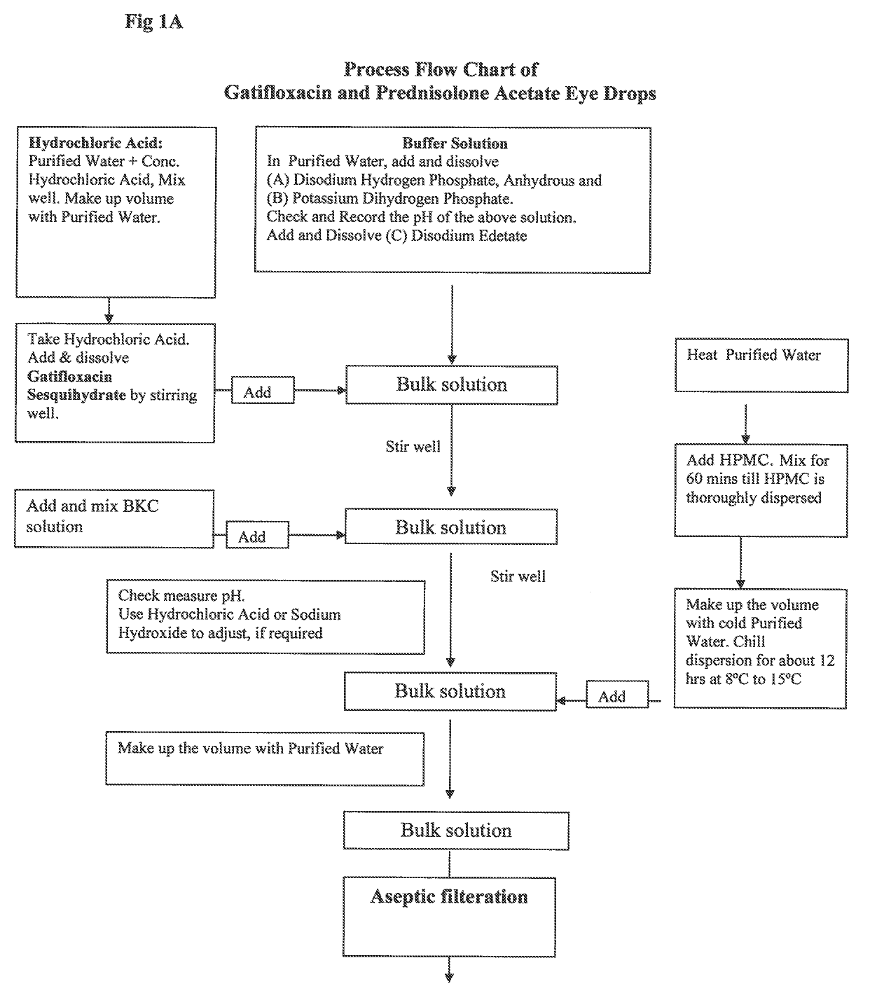

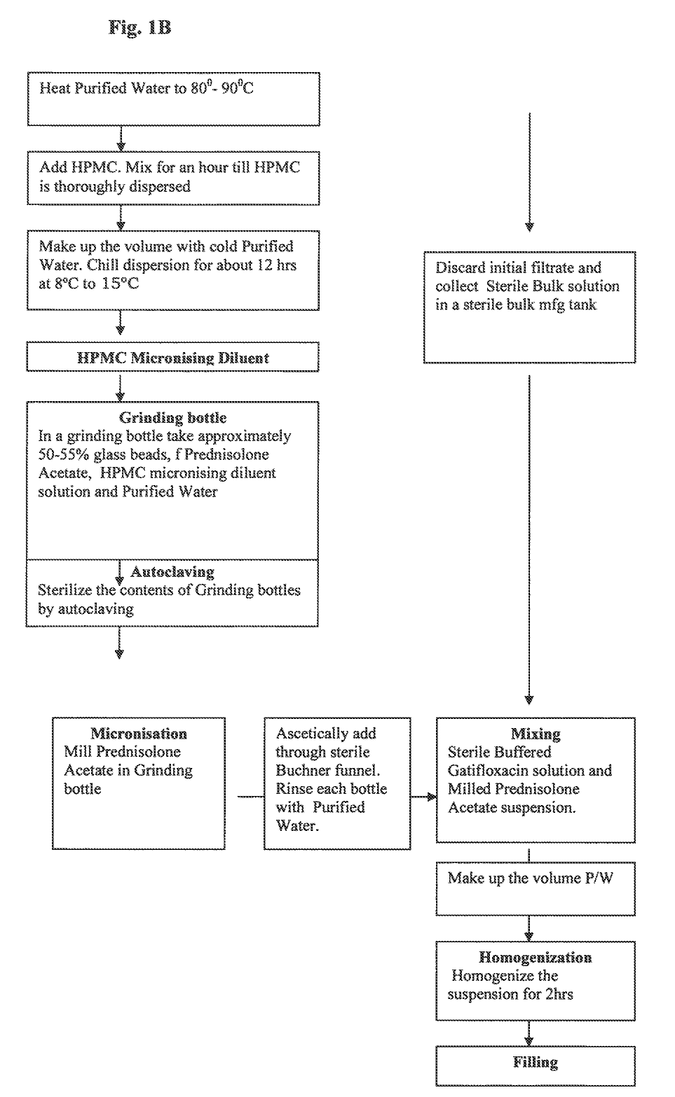

### DETAILED DESCRIPTION OF THE INVENTION

The bactericidal mechanism of action of fluoroquinolones, including gatifloxacin, is different than those of the aminoglycosides, macrolides and tetracycline antibiotics. Therefore, gatifloxacin can be effective/active against pathogenic agents resistant to these antibiotics and these antibiotics can be active against pathogenic agents resistant to gatifloxacin. No cross-resistance has been observed among gatifloxacin and previous mentioned classes of antibiotics. Cross-resistance has been observed among systemic gatifloxacin and some other fluoroquinolones.

Gatifloxacin (4^{th} generation fluoroquinolone, approved in 2003 as a 0.3% solution called Zymar®, marketed by Allergan), shows a broad spectrum of antibacterial activity against gram-positive and gram-negative microorganisms, anaerobic organisms, mycobacteria and species of Mycoplasma, and Chlamydia. Several studies have been conducted comparing the antibacterial activity of gatifloxacin to the activity of other classes of antibiotics and other fluoroquinolones. In vitro studies showed that gatifloxacin is more powerful than other quinolones, including ciprofloxacin, ofloxacin, levofloxacin and moxifloxacin against gram-positive microorganisms such as methicillin resistant Staphylococcus aureus, and Staphylococcus epidermidis. Gatifloxacin was eight times more effective than ofloxacin against Streptococcus epidermidis and showed increased activity compared to ofloxacin against many gram-negative microorganisms. Gatifloxacin was two times more effective than ofloxacin against Haemophylus influenzae clinical isolated cases. In other studies, gatifloxacin was four times more effective/active than ciprofloxacin against mycobacterium, was more effective than ciprofloxacin (p<0.001) against Mycbacterium chelonae and showed a synergistic action when combined with amicacin and claritromicin. Besides these studies, one other study was conducted comparing the activity of gatifloxacin with that of moxifloxacin, ciprofloxacin and ofloxacin in vitro. Investigators concluded that the sensitivity profiles achieved by the 4^{th} generation fluoroquinolones (gatifloxacin and moxifloxacin) can offer advantages over 2^{nd} generation fluoroquinolones (ciprofloxacin and ofloxacin). In vitro studies showed that the bacterial resistance to gatifloxacin is low.

Bacterial resistance rates regarding resistant mutant strains of Pseudomonas aeruginosa and Escherichia coli for gatifloxacin are similar to ciprofloxacin and norfloxacin. However, bacterial resistance rates for Staphylococcus aureus and Staphylococcus epidermidis are lower for gatifloxacin than for ciprofloxacin or norfloxacin. Gatifloxacin was also less affected by topoisomerase II or gyrase mutations, when compared to ofloxacin, ciprofloxacin, sparfloxacin and trovafloxacin.

The ophthalmic composition of the present invention is contained in pharmaceutically acceptable excipients and vehicles. The excipients used in the manufacturing of the current invention are Methocel (F4M) HPMC IP (Indian Pharmacopeia) as suspending agent; Disodium Hydrogen Phosphate as buffer salt; Potassium Dihydrogen Phosphate BP as buffer salt; Disodium Edetate IP as chelating agent; Hydrochloric Acid 0.5 N as solbuliser for gatifloxacin; Sodium Hydroxide for pH adjustment; hydrochloric acid for pH adjustment and purified water as a vehicle.

The composition of the present invention has a pH in the range of 6.5 to 7.4. The ophthalmic composition has been formulated to have osmotic values that are compatible with the aqueous humor of the eye and ophthalmic tissues. Such osmotic values will generally be in the range of from about 250 to about 350 milliosmoles per kilogram of water (“mOsm/kg”). The formulation is designed to have particle size wherein 95% of particles are less than 7 micron and no particle more than 10 micron. The formulation also has a weight per ml in the range of 0.9 g/ml and 1.1 g/ml.

Ophthalmic pharmaceutical product is packaged in a multidose form. Preservatives are thus required to prevent microbial contamination during use. Suitable preservatives include benzalkonium chloride and other agents known to those skilled in the art. In the current formulation benzalkonium chloride USP 0.05 mg (0.005% w/v) is used as an effective preservative.

The present composition can be provided in a kit containing 1-10 ml plastic dropper bottles designed for topical administration of ophthalmic solution. A 5 ml plastic dropper is preferred.

### Pharmacokinetic Data

In relation to gatifloxacin pharmacokinetics, a study was conducted whereby gatifloxacin ophthalmic solution (0.3% or 0.5% w/v) was instilled in one of the eyes of 6 healthy male individuals in a dose-escalation regimen, beginning with a single dose of 2 drops, followed by 2 drops, 4 times a day, for 7 days; and finally, 2 drops, 8 times a day, for 3 days. During the assessment period, the plasma levels of gatifloxacin remained below the inferior limit of quantification (5 ng/ml) in all individuals who participated in the study, showing that the concentrations of gatifloxacin were acceptably low after application to the eye and that it would have little to no effect on the systemic safety profile. On the other hand, there have been studies comparing the penetration of gatifloxacin in the eye structures, when compared the ciprofloxacin, has been observed that the gatifloxacin has better penetration in the tissue (p<0.005). Solomon R, Donnenfeld E D, Perry H D, Snyder R W, Nedrud C, Stein J, Bloom A. “Penetration of topically applied gatifloxacino 0.3%, moxifloxacin 0.5%, and ciprofloxacin 0.3% into the aqueous humor.” Ophthalmology, 2005; 112(3):466-469.

A study comparing the pharmacodynamic of gatifloxacin and ciprofloxacin showed the impact of gatifloxacin pharmacokinetics in relation to ciprofloxacin on the drugs' antimicrobial effects. Gatifloxacin showed a longer half-life than ciprofloxacin and the results of this study showed the important role of a longer half-life, resulting in a superior antimicrobial effect. Vostrov S N, Kononenko O V, Lubenko I Y, Zinner S H, Firsov A A. “Comparative pharmacodinamics of gatifloxacin and ciprofloxacin in an in vitro dynamic model: prediction of equiefficient doses and the breakpoints of the area under the curve/MIC ratio.” Antimicrob Agents Chemother 2000; 44(4):879-884.

### Clinical Aspects

Gatifloxacin

Results of previous pre-clinical and clinical studies showed that gatifloxacin in an ophthalmic solution is clinically safe and effective in the treatment of infections caused by bacteria. The 0.3% gatifloxacin at ophthalmic solution Zymar® was shown to be effective in the treatment of acute bacterial conjunctivitis in children and adults, including elderly people. In a randomized, double-blind, multicenter clinical study, in which the patients were treated for five days, Zymar® ophthalmic solution was superior to the vehicle between days 5 and 7 in the treatment of patients with bacterial conjunctivitis confirmed by culture. The results of the clinical study showed a clinical cure rate for 77% of the group treated with gatifloxacin compared to 58% to the group treated with placebo. The microbiological results in this study showed a statistically significant superiority of gatifloxacin, and the killing rate of etiological agents for gatifloxacin was 92% versus 72% for placebo. In active-controlled comparative study, the rates of microbiological cure on day 6 of treatment were similar for gatifloxacin (83.3%) and ofloxacin (85.3%).

In one other randomized, double-blind study comparing gatifloxacin to ciprofloxacin in patients with keratitis, the group treated with gatifloxacin showed a significantly higher rate of complete ulcer healing than the group treated with ciprofloxacin. The action of gatifloxacin against gram-positive cocci was significantly superior to that of the ciprofloxacin (p<0.001), and the percentage of ulcers caused by these healed pathogens in the gatifloxacin group was significantly higher in the group treated with gatifloxacin (p=0.009). The authors concluded that the gatifloxacin can be a preferred alternative in relation to ciprofloxacin in the treatment of bacterial keratitis.

The efficacy and safety of gatifloxacin 0.3% and levofloxacin 0.5% ophthalmic solutions were compared in a prospective study in which patients received treatment for one week, in the pre-operative period of a cataract surgery and the results have shown that the rate of bacterial reduction in the conjunctiva was 74.3% (55/74 individuals) for gatifloxacin and 70.0% (42/60 individuals) for levofloxacin. None of the individuals treated develop a post-operative infection (endophthalmitis), which shows that both substances were effective in the reduction of bacterial flora in the eye, and may be safely indicated for the prophylactic treatment in the pre-operative period.

The most commonly isolated bacteria in clinical studies conducted with Zymar® were Staphylococcus aureus, Staphylococcus epidermidis and Streptococcus pneumoniae. However, the varieties of bacteria observed during the studies include all species commonly reported as etiological agents of conjunctivitis. In vitro tests of 379 isolated microorganisms showed that there was 95.8% sensitiveness to gatifloxacin on the first day of treatment. All Streptococcus pneumoniae isolates were sensitive to gatifloxacin in all study visits both in relation to gatifloxacin and the controls. All gram-negative bacteria were also sensitive to gatifloxacin in all study visits both in relation to gatifloxacin and controls, including all Haemophilus influenzae isolates.

The adverse events reported during the clinical studies conducted with gatifloxacin were similar to those reported for other ophthalmic preparations containing fluoroquinolones. There were no statistically significant differences between gatifloxacin and the active control ofloxacin or placebo in terms of frequency, type, intensity or relation to the causes of adverse events. The ophthalmologic exams and the visual acuity assessment were also similar among the patients treated with the active substances and the placebo. The phase III clinical studies, which evaluated the systemic exposition of gatifloxacin in an ophthalmic solution 0.3% (Zymar®) did not detect the effects described for the quinolone class including phototoxic or allergic reactions, as well as cardiovascular, articular or tendinous/sinewy diseases. Adverse events in the central nervous system were rare.

It has been concluded that the gatifloxacin ophthalmic solution 0.3% (Zymar®) for topical use shows an excellent safety profile with low systemic exposure and without evidence of the effects of quinolone class. Therefore, the properties described for gatifloxacin justify the choice of its inclusion in the composition of the present invention in combination with prednisolone for topic ocular use.

Prednisolone

Prednisolone acetate is 21-acetate of 11 β, 17, 21-triidroxipregna-1,4 dieno-3,20 diona substance that suppress the inflammatory response in relation to a variety of agents that slow the healing process. Prednisolone is a corticosteroid and as such may prevent or suppress inflammation in response to multiple events, including infectious, chemical, radioactive, mechanical and immunological stimuli. Although the use of corticosteroids as anti-inflammatories is not aimed to treat the primary cause of the disease, the suppression of inflammation is extremely useful in clinical terms. Multiple mechanisms are involved in the suppression of inflammation by glucocorticoids, and it is understood today that glucocorticoids inhibit the production of certain inflammatory factors by various cells which cause an inflammatory response. As a result, use of prednisolone results in the reduction in the release of vasoactive and chemical active factors, the reduction of lipolytic and proteolytic enzyme secretion and the reduction of leukocyte outflow to damaged areas and reduction of fibrosis. Thus, in relation to the eye, the anti-inflammatory response comprises a series of inhibitory phenomena on exudation, hiperemia, cell infiltration, fibroblastic activity, epithelial and endothelial regeneration, neo-vascularization and capillary permeability in the eye structures.

While prednisolone acetate ophthalmic suspension is contraindicated in most viral diseases of the cornea and conjunctiva including epithelial herpes simplex keratitis (dendritic keratitis), vaccinia, varicella, mycobacterial infection of the eye and fungal diseases of ocular structures, it has been surprisingly found that that when combined with gatifloxacin at 0.3% w/v, the overall risk benefit of use of prednisolone in combination in indicated conditions is highly favorable.

The combination of gatifloxacin and prednisolone in a single composition in the concentrations stated herein is novel and has characteristics which may be considered similar in concept to traditional combination therapies, but demonstrates superior properties to other combination ophthalmic products.

### Clinical Data:

Post marketing data of the use of the combination was obtained by a physician experience questionnaire. The objective of the physician experience questionnaire was to evaluate the opinion of ophthalmologists' for safety, efficacy and compliance of the combination. The second objective was to have physician experience on the present formulation versus existing similar combination or individual treatment. The data collection is still ongoing. Data obtained from 102 physicians has been compiled and is presented below. The composition referred to as Z-Pred is the 0.3% w/v gatifloxacin and 1.0% w/v prednislone combination with benzalkonium as a preservative as described herein.

Methodology: The following questions were used to access physician's opinion on the combination.
1. Have you prescribed Z-Pred? Answers were obtained as Yes/No.
2. In your clinical experience how would you rate the effectiveness of Z-Pred in the treatment of steroid responsive inflammatory ocular conditions? The ratings were obtained on a five point rating scale: very effective, effective, neither effective nor ineffective, ineffective and not at all effective.
3. Do you use Z-Pred in cases such as pre & post surgical where a corticosteroid is indicated & where bacterial infection or a risk of bacterial ocular infection exist? Answers were obtained as Yes/No.
4. What has been your level of satisfaction in above cases (Q. No.3)? The ratings were obtained on a five point rating scale: very satisfactory, satisfactory, neither satisfactory nor dissatisfactory, dissatisfactory and very dissatisfactory.
5. In your clinical experience have you seen any major adverse events seen with the use of Z-Pred? Answers were obtained as Yes/No.
6. Would you rate Z-Pred better than the similar existing anti-infective & steroid fixed dose combinations? Answers were obtained as Yes/No.
7. Do you see a better patient compliance with Z-Pred as compared to individual drug therapy? Answers were obtained as Yes/No.
   Results: Result of the questionnaire from 102 physicians is presented in table below.

TABLE II

| Sl. | | | N | |
|---|---|---|---|---|
| No | Parameters | Assessment Criteria | 102 | (%) |
| 1 | Have you prescribed Z-Pred? | Yes | 101 | 99.0 |
| | | No | 1 | 1.0 |
| 2 | In your clinical experience how would you rate the effectiveness of | Very effective/Effective | 101 | 99.0 |
| | Z-Pred in the treatment of steroid responsive inflammatory ocular | Neither effective nor ineffective | 1 | 1.0 |
| | conditions? | Ineffective | 0 | 0.0 |
| | | Not at all eff | 0 | 0.0 |
| 3 | Do you use Z-Pred in cases such as pre & post surgical where a | Yes | 87 | 85.3 |
| | corticosteroid is indicated & where bacterial infection or a risk of | No | 14 | 13.7 |
| | bacterial ocular infection exist? | | | |
| 4 | What has been your level of Satisfaction in above cases (Q. No. 3)? | Very satisfactory/Satisfactory | 92 | 90.2 |
| | | Neither satisfactory nor dissatisfactory | 2 | 2.0 |
| | | Dissatisfactory | | 0.0 |
| | | Very dissatisfactory | | 0.0 |
| 5 | In your clinical experience have you seen any major adverse events | Yes | 6 | 5.9 |
| | seen with the use of Z-Pred? | No | 96 | 94.1 |
| 6 | Would you rate Z-Pred better than the similar existing anti-infective | Yes | 92 | 90.2 |
| | & steroid fixed dose combinations? | No | 6 | 5.9 |
| 7 | Do you see a better patient compliance with Z-Pred as compared to | Yes | 101 | 99.0 |
| | individual drug therapy? | No | 1 | 1.0 |

### Results:

In the survey, it was observed that most physicians prescribed the combination in their clinical practice (99%). As shown in Table I, most physicians rated the combination as very effective or effective for (99%) for treatment of steroid responsive ocular conditions. Similarly, 85.3% physicians used the combination for pre- and post-surgical cases where a corticosteroid was indicated and where bacterial infection or risk of bacterial infection existed. The level of satisfaction with the combination was generally high, with 90.2% physicians rating their level of satisfaction as either very satisfactory or satisfactory (combined percentage being 90.2%). There were 2% of total cases who answered as neither satisfactory nor dissatisfactory. None rated the treatment as dissatisfactory or very dissatisfactory. Most physicians rated the combination as better than existing anti-infective and steroid fixed dose combinations (90.2%). Perhaps most importantly, ninety nine percent (99%) physician rated a better patient compliance as compared to individual drug therapy. The physician experience questionnaire outcome supports the scientific claim that the formulation of topical ocular gatifloxacin plus prednisolone offers a good alternative over existing steroids and anti-infective drugs with added patient compliance.

1) A topical ophthalmic composition for treating or preventing ophthalmic bacterial infections in a human patient wherein the ophthalmic composition contains gatifloxacin and prednisolone.

2) The composition of claim 1 wherein the composition is also useful in preventing inflammation and bacterial infection in the eye following ophthalmic surgical procedures.

3) The composition of claim 1 wherein the concentration of gatifloxacin is 0.01% to 1.5% w/v.

4) The composition of claim 1 wherein the concentration of prednisolone is 0.01% w/v-2.0% w/v.

5) The composition of claim 3 wherein the concentration of gatifloxacin is 0.1% w/v-0.5% w/v.

6) The composition of claim 4 wherein the concentration of prednisolone is 0.1% w/v-1.5% w/v.

7) The composition of claim 5 wherein the concentration of gatifloxacin is 0.3% w/v.

8) The composition of claim 5 wherein the concentration of prednisolone is 1.0% w/v.

9) The composition of claim 1 wherein the concentration of gatifloxacin is 0.1% w/v-0.5% w/v and the concentration of prednislone is 0.1% w/v-1.5% w/v.

10) The composition of claim 9 wherein the concentration of gatifloxacin is 0.3% w/v and the concentration of prednislone is 1.0% w/v.

11) The composition of claim 10 further comprising benzalkonium chloride.

12) The composition of claim 11 wherein the benzalkonium chloride is present in the amount of 0.005% w/v.

13) The composition of claim 10 wherein the pH is in the range of 6.5 to 7.4.

14) The composition of claim 1 wherein the composition has an osmotic value from 250 to 350 milliosmoles per kilogram of water.

15) The composition of claim 10 wherein the composition is provided as a kit containing a 1-10 ml plastic dropper designed for topical administration of the composition.

16) The composition of claim 10 wherein the composition is provided as a kit containing a 5 ml plastic dropper designed for topical administration of the composition.

17) The composition of claim 10 wherein the composition may be used for treatment of bacterial conjunctivitis by applying 1-4 drops of the composition to each eye per day.

18) The composition of claim 10 wherein the composition may be used for prevention and treatment of inflammatory conditions following eye surgery by applying 1-4 drops of the composition to each eye per day.

19) The composition of claim 10 wherein the composition may be used for prevention and treatment of inflammatory conditions before or following eye surgery by applying 1-4 drops of the composition to each eye per day.

20) The composition of claim 10 wherein the composition may be used for prevention of bacterial conjunctivitis following eye surgery by applying 1-4 drops of the composition to each eye per day.
